(19) United States
(12) Patent Application Publication
(10) Pub. No.: US 2025/0050033 A1
(43) Pub. Date: Feb. 13, 2025

(54) SUB-ASSEMBLY OF A MEDICAMENT DELIVERY DEVICE

(71) Applicant: SHL Medical AG, Zug (CH)

(72) Inventors: Thad Miley, Deerfield Beach, FL (US); Aaron Raspler, Deerfield Beach, FL (US)

(21) Appl. No.: 18/718,494

(22) PCT Filed: Dec. 2, 2022
(86) PCT No.: PCT/EP2022/084168

§ 371 Date: Jun. 11, 2024

Related U.S. Application Data
(60) Provisional application No. 63/290,411, filed on Dec. 16, 2021.

(30) Foreign Application Priority Data
Jan. 18, 2022 (EP) 22151958.0

(51) Int. Cl. A61M 5/32; A61M 5/20

(52) U.S. Cl. CPC A61M 5/326; A61M 5/2033; A61M 2005/2006

(57) ABSTRACT

The present disclosure relates to a sub-assembly of a medicament delivery device comprising a plunger rod configured to be moveable inside the medicament delivery device, a spring abutting the plunger rod in one end and abutting an inner wall of the sub-assembly in another end to compress the spring upon the plunger rod being moved towards a distal end of the medicament delivery device, an arm extending axially and being immovable with respect to a housing, the arm having a radially extending end section, an inner surface of which end section pressing radially against the spring-loaded plunger rod to axially fix the spring-loaded plunger rod relative to the arm, a rear wall member axially movable from a lock position where an inner wall of the rear wall member radially encloses an outer surface of the arm to a release position where the rear wall member is radially offset relative to the outer surface of arm, a radially flexible tab having an axially directed surface adjacent to a counter surface of an engagement member to form an axial abutment preventing the rear wall member from moving into the release position, and a release member configured to be axially moved into contact with a radial protrusion of the flexible tab to radially deflect the flexible tab, thereby radially offsetting the axially directed surface from the counter surface of the engagement member.

### CROSS-REFERENCE TO RELATED APPLICATIONS

The present application is a U.S. National Phase Application pursuant to 35 U.S.C. § 371 of International Application No. PCT/EP2022/084168 filed Dec. 2, 2022, which claims priority to (i) U.S. Provisional Patent Application No. 63/290,411 filed Dec. 16, 2021, and (ii) European Patent Application No. 22151958.0 filed Jan. 18, 2022. The entire disclosure contents of these applications are herewith incorporated by reference into the present application.

### TECHNICAL FIELD

The present disclosure relates to a sub-assembly of a medicament delivery device.

### BACKGROUND

Prior art medical delivery devices, such as an auto-injectors, are commonly designed with multiple automatic functions. For example, instead of asking a user to manually push a plunger rod for expelling a medicament, in these prior art auto-injectors, a plunger rod is held in a position where a spring utilized to spring-load the plunger rod is in a compressed state. To release the plunger rod, a user needs to push a release member to release the plunger rod for expelling the medicament. The release member can be a needle cover that is telescopically arranged related to a housing of the medicament delivery device, or a button arranged at a distal end or a side of the housing of the medicament delivery device.

To avoid an unintentionally releasing of the plunger rod, e.g. due to shaking or dropping of the medicament delivery device, WO200911454A1 discloses an auto-injector with a safety clip that must be removed by the user before medicament can be delivered to the user. As a result, an undesired operational step during administration of medicament is added. There is thus room for improvement.

### SUMMARY

One objective is to solve, or at least mitigate, this problem in the art and to provide an improved sub-assembly of a medicament delivery device.

This objective is attained in an aspect by a sub-assembly of a medicament delivery device comprising: a tubular housing, a plunger rod, a spring, a rear wall member, a radially flexible tab, an arm, and a release member; the plunger rod is configured to be moved by the spring inside the housing to expel a medicament of the medicament delivery device; the arm is axially immovable with respect to the housing, the arm having a radially extending end section, an inner surface of which end section pressing radially against the plunger rod to axially fix the plunger rod relative to the arm; the rear wall member is axially movable from a lock position where an inner wall of the rear wall member radially encloses an outer surface of the arm (so as to restrict the arm from moving radially outwards) to a release position where the rear wall member is radially offset relative to the outer surface of the arm (so as to allow the arm to move radially outwards; thereby releasing the plunger rod to move axially (in the proximal direction) relative to the arm); the radially flexible tab comprises an axially directed surface adjacent to a counter surface of an engagement member to form an axial abutment preventing the rear wall member from moving into the release position; the release member is configured to be axially moved into contact with a radial protrusion of the flexible tab to radially deflect the flexible tab, thereby radially offsetting the axially directed surface from the counter surface of the engagement member.

An advantage of this solution is that the rear wall member is held in the lock position by the radially flexible tab which holds the rear wall member such that the rear wall member is axially immovable. The release member, being e.g. a needle cover, will slide in a distal direction and into contact with the radially flexible tab to push the rear cap out of the lock position for plunger rod spring release.

Thus, with the radially flexible tab, there is no need for the safety clip utilized in the prior art and the user is relieved from the burden of having to remove the safety clip before using the device to deliver the medicament.

In an embodiment, the release member is configured to be further axially moved to move the rear wall member to the release position after the axially directed surface has been radially offset from the counter surface of the engagement member.

In an embodiment, an actuator housing comprising an end wall with an interior against which said another end of the spring abuts and an opening via which the plunger rod extends for contacting the arm, the actuator housing further comprising the radially flexible tab and being immovable with respect to the plunger rod upon the rear wall member being in the lock position, and wherein the rear wall member comprises the engagement member.

In an embodiment, the actuator housing comprises the arm extending axially from an exterior of the end wall of the actuator housing.

In an embodiment, the housing comprises the arm extending axially from an interior of an end wall of the housing.

In an embodiment, the plunger rod comprises an indentation being received within the inner surface of the end section of the arm.

In an embodiment, the rear wall member comprises the radially flexible tab and the housing comprises the engagement member arranged at an interior of a side wall of the housing.

In an embodiment, the release member comprises a needle cover. Optionally, the needle cover is configured to axially move into contact with the radial protrusion of the flexible tab to radially deflect the flexible tab upon a user pressing a proximal end of the needle cover towards a site of the body for medicament delivery. Optionally, the needle cover is configured to axially move into contact with the rear wall member to move the rear wall member axially (specifically in the distal direction) relative to the arm upon a user pressing a proximal end of the needle cover towards a site of the body for medicament delivery.

An aspect concerns a medicament delivery device comprising any sub-assembly as described above. Optionally, the medicament delivery device is an auto-injector. Optionally, the auto-injector is a needle-based injector or a needleless injector. Optionally, the medicament delivery device comprises a medicament container (such as a syringe or a cartridge) that is immovable relative to the tubular housing.

Generally, all terms used in the claims are to be interpreted according to their ordinary meaning in the technical field, unless explicitly defined otherwise herein. All references to “a/an/the element, apparatus, component, means, step, etc.” are to be interpreted openly as referring to at least one instance of the element, apparatus, component, means, step, etc., unless explicitly stated otherwise. The steps of any method disclosed herein do not have to be performed in the exact order disclosed, unless explicitly stated.

### BRIEF DESCRIPTION OF THE DRAWINGS

Aspects and embodiments are now described, by way of example, with reference to the accompanying drawings, in which:

FIG. 1 shows a perspective view of a prior art medicament delivery device;

FIG. 2 shows individual components of the prior art injector of FIG. 1 in an exploded view;

FIG. 3 shows a perspective view of a medicament delivery device in which a sub-assembly according embodiments may be implemented;

FIG. 4 shows individual components of the injector of FIG. 3 in an exploded view according to a first embodiment;

FIG. 5 shows individual components of the injector of FIG. 3 in an exploded view according to a second embodiment;

FIGS. 6-8 show the rear wall member being pushed into the release position according to the first embodiment;

FIG. 9 illustrates a radially flexible tab according to the first embodiment;

FIGS. 10-12 show the rear wall member being pushed into the release position according to the second embodiment;

FIG. 13 illustrates a radially flexible tab according to the second embodiment;

FIG. 14 illustrates a needle cover in a first position according to an embodiment;

FIG. 15 illustrates a needle cover in a second position according to an embodiment; and

FIG. 16 illustrates an actuator housing according to an embodiment.

### DETAILED DESCRIPTION

The aspects of the present disclosure will now be described more fully hereinafter with reference to the accompanying drawings, in which certain embodiments of the invention are shown.

These aspects may, however, be embodied in many different forms and should not be construed as limiting; rather, these embodiments are provided by way of example so that this disclosure will be thorough and complete, and to fully convey the scope of all aspects of invention to those skilled in the art. Like numbers refer to like elements throughout the description.

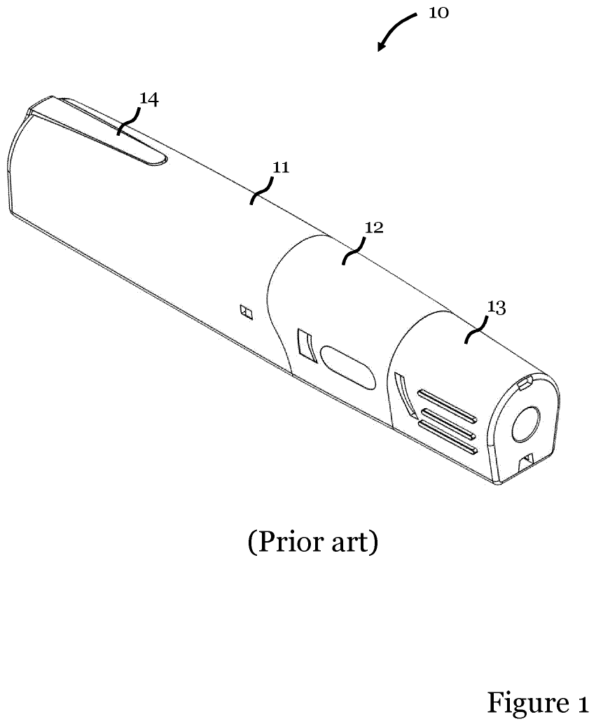

FIG. 1 shows a perspective view of a prior art medicament delivery device 10. The particular medicament delivery device 10 illustrated in FIG. 1 is an injector configured to inject a dose of medicament into the body of a user via a syringe arranged in a back outer shell 11 and front outer shell 12 of the injector 10 after a protective front cap 13 has been removed.

The prior art injector 10 is further arranged with a safety clip 14 which must be removed by the user before use of the injector 10, since a spring-loaded plunger rod arranged in the injector 10 otherwise cannot be released to cause medicament delivery.

An end of the injector 10 facing towards the user is referred to as proximal end while an end of the injector 100 facing away from the user is referred to as distal end.

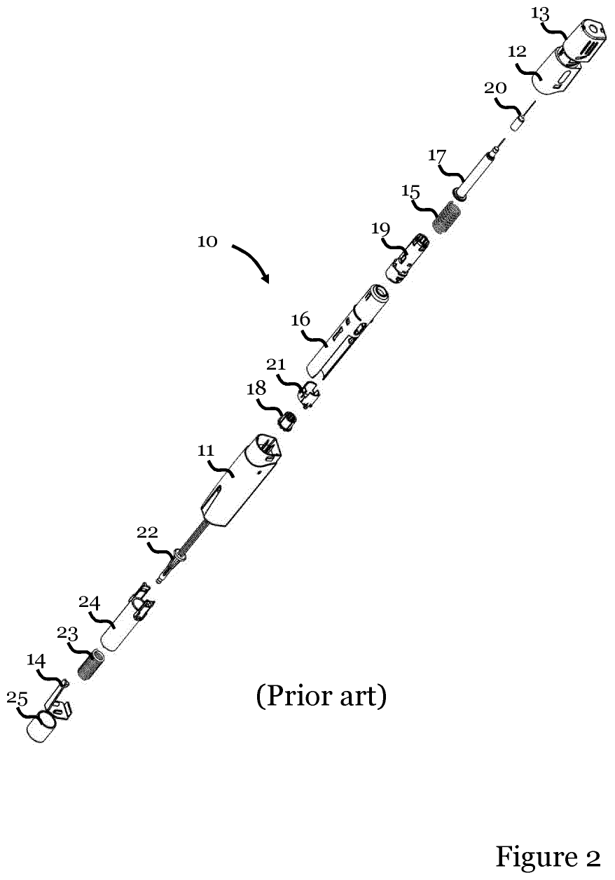

FIG. 2 shows individual components of the prior art injector 10 of FIG. 1 in an exploded view. The back outer shell 11 and the front outer shell 12 house all components except for the safety clip 14 which is attached to an exterior of the back outer shell 11, while the front cap 13 is snapped into attachment with a front end of the front outer shell 12 for protecting the user from a syringe as will be described in more detail in the following.

The injector 100 comprises a needle cover spring 15 arranged to spring-load a needle cover 16 utilized to protect the user from the syringe 17. Further associated with the syringe 17 is a syringe support 18 and a syringe carrier 19 (and medicament 20 to be delivered by the injector 10). A lock out collar 21 is employed to prevent the needle cover 16 from again being pushed towards a distal end position once medicament has been delivered and the needle cover 16 has reverted to a position at the proximal end of the injector 10 for covering the syringe 17.

The injector 10 further comprises a plunger rod 22 being spring-loaded with a plunger rod spring 23 against a distal end wall of an actuator housing 24 for pushing the medicament 20 through the syringe 17 for delivery to the user upon plunger rod spring release.

A rear cap 25 is axially movable from a lock position in which the plunger rod 22 is held in a position where the plunger rod spring 23 is compressed to a release position where the rear cap 25 is moved by the needle cover 105 towards a distal end, and the plunger rod spring 23 is released, thereby causing spring release to have the plunger rod 22 push the medicament 20 through the syringe 17 for delivery. However, to cause spring release, the user must first remove the safety clip 14 which holds the rear cap 25 in the lock position when being attached to the back outer shell 11.

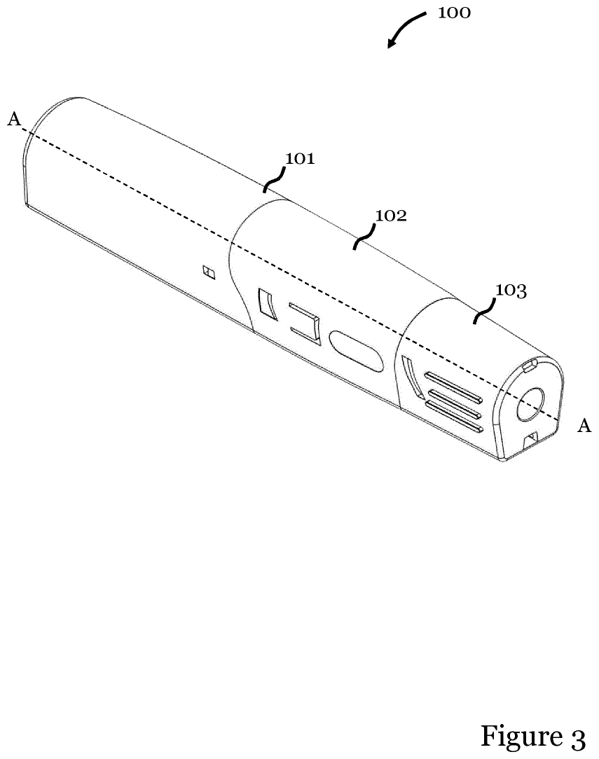

FIG. 3 shows a perspective view of a medicament delivery device 100 in which a sub-assembly according embodiments may be implemented. The particular medicament delivery device 100 illustrated in FIG. 3 is an injector configured to inject a dose of medicament into the body of a user via a syringe arranged in a back outer shell 101 and front outer shell 102 of the injector 100 after a protective front cap 103 has been removed.

An end of the injector 100 facing towards the user is referred to as proximal end while an end of the injector 100 facing away from the user is referred to as distal end. The injector 100 extends along an axis A between the proximal end and the distal end.

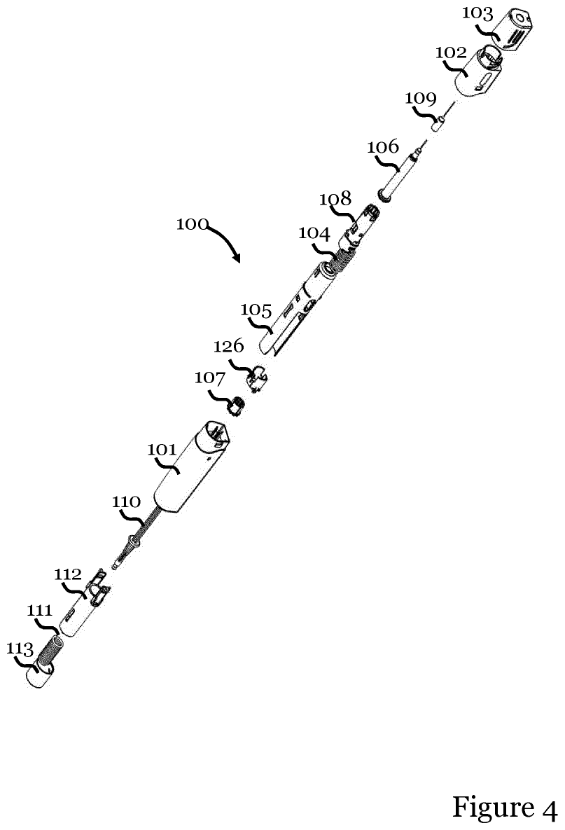

FIG. 4 shows individual components of the injector 100 of FIG. 3 in an exploded view according to a first embodiment. The back outer shell 101 and the front outer shell 102 house all components, while the front cap 103 is snapped into attachment with a front end of the front outer shell 102 for protecting the user from a syringe as will be described in more detail in the following.

The injector 100 comprises a needle cover spring 104 arranged to spring-load a needle cover 105 utilized to protect the user from the syringe 106. Further associated with the syringe 106 is a syringe support 107 and a syringe carrier 108 (and medicament 109 to be delivered by the injector 100).

The injector 100 further comprises a plunger rod 110 being spring-loaded with a plunger rod spring 111 against a distal end wall of an actuator housing 112 for pushing the medicament 109 through the syringe 106 for delivery to the user upon plunger rod spring release.

A rear cap 113 is axially movable from a lock position in which the plunger rod 110 is held in a position where the plunger rod spring 111 is fully compressed to a release position where the rear cap 113 is moved by the needle cover 105 towards a distal end, and the plunger rod spring 111 is released, thereby causing spring expansion to have the plunger rod 110 push the medicament 109 through the syringe 106 for delivery.

Thus, the rear cap 113 locks the plunger rod 111 in order to prevent accidental release of the plunger rod spring 111. Plunger rod release and subsequent medicament delivery can only be achieved upon the user removing the front cap 103 and pressing a proximal end of the needle cover 105 against a site of the body where the medicament is to be injected. The needle cover 105 will thus slide in a distal direction and push the rear cap 113 out of the lock position for plunger rod spring release. A lock-out collar 126 is further used to block distal movement of the needle cover 105 once the medicament has been delivered.

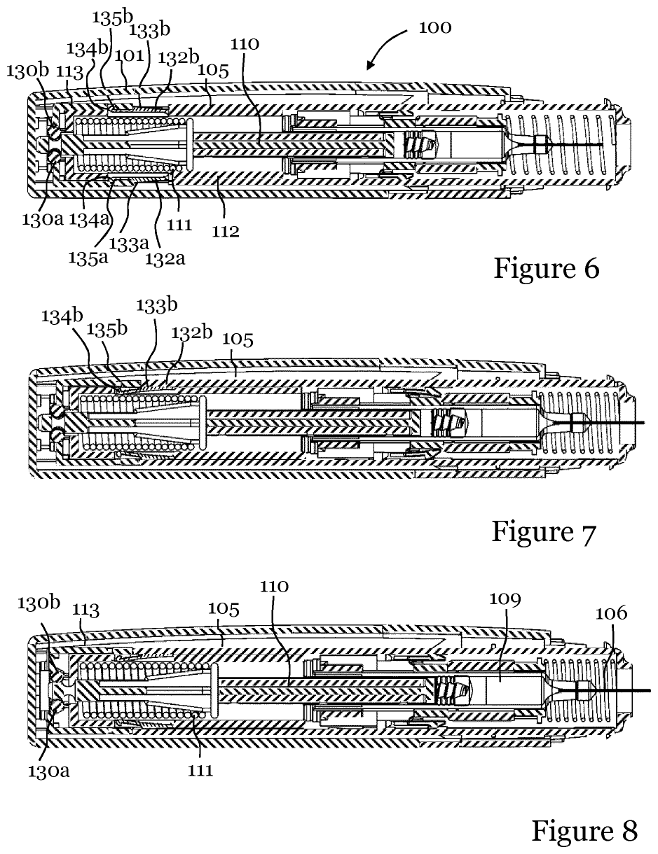

An advantage of this solution is that the rear cap 113 is held in the lock position by a radially flexible tab which holds the rear cap 113 such that the cap is axially immovable. The radially flexible tab is in this embodiment arranged on the actuator housing 112 and engages with a counter-engagement member of the rear cap 113 as will be described in FIGS. 6-9. The needle cover 105 will thus slide in a distal direction and into contact with the radially flexible tab to push the rear cap 113 out of the lock position for plunger rod spring release.

Thus, with the radially flexible tab, there is no need for the safety clip 14 of the prior art injector 10 and the user is relieved from the burden of having to remove the safety clip 14 before using the injector to deliver the medicament.

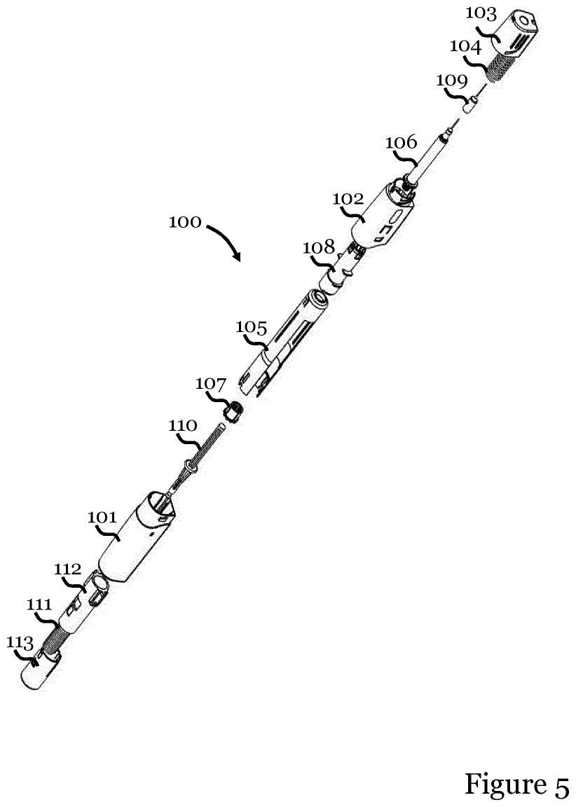

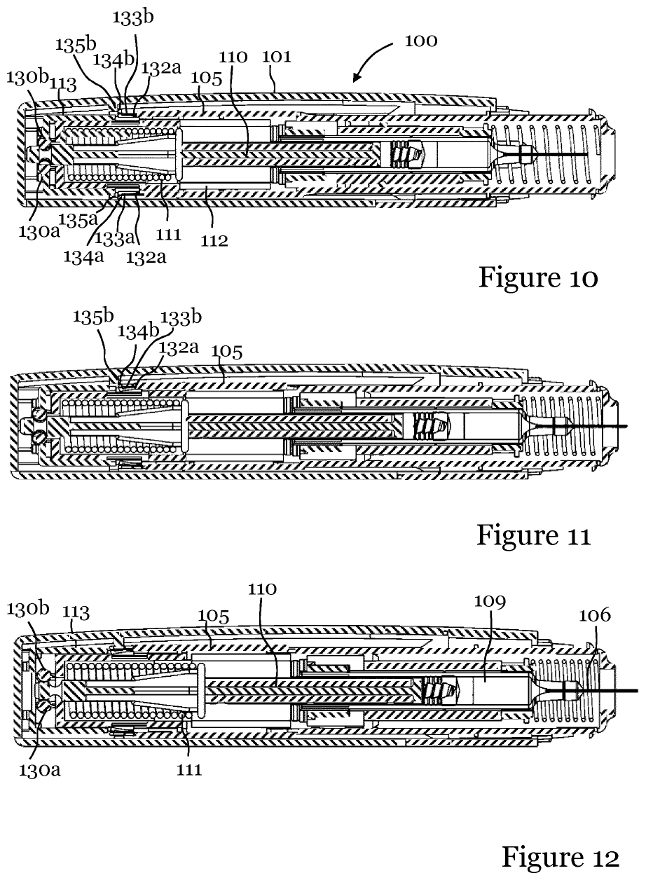

FIG. 5 shows individual components of the injector 100 of FIG. 3 in an exploded view according to a second embodiment. As can be seen, the components of the second embodiment are the same as those of the first embodiment illustrated in FIG. 4, with the exception of the lock-out collar 126. In the second embodiment of FIG. 5, the radially flexible tab is arranged on the rear cap 113 and engages with a counter-engagement member of a housing as will be described in FIGS. 10-13.

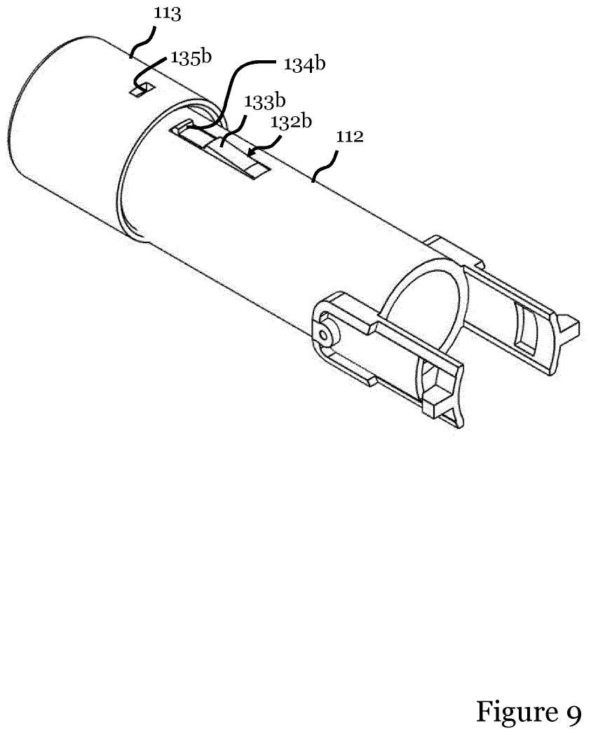

FIGS. 6-8 illustrate sectional views of the injector 100 of FIG. 3 taken along line A-A according to the first embodiment, while FIG. 9 shows the actuator housing comprising the radially flexible tab in accordance with the first embodiment illustrated in FIG. 4. Only a few components of the exploded view of FIG. 4 will be discussed.

With reference to FIG. 6, in the sub-assembly of this embodiment, the plunger rod 110 configured to be moveable inside the injector 100 is spring loaded by the spring 111 abutting the plunger rod 110 in one end and abutting an inner wall of the sub-assembly in another end, in this example a distal inner wall of the actuator housing 112, to compress the spring 111 upon the plunger rod 110 being moved towards a distal end of the injector 100.

Further, the sub-assembly comprises at least one arm (in this first exemplifying embodiment two arms 130a, 130b) extending axially and being immovable with respect to a housing. In an example where the sub-assembly is used with the medicament delivery device of FIG. 3, the housing is the back outer shell 101. The plunger rod 110 extends axially through an opening of the actuator housing 112 for contacting the arms 130a, 130b.

The arms 130a, 130b have a radially extending end section, an inner surface of which end section presses radially against the spring-loaded plunger rod 110 to axially fix the spring-loaded plunger rod 110 relative to the arms 130a, 130b.

In this embodiment, the actuator housing 112 comprises the arms 130a, 130b extending axially from an exterior of the end wall of the actuator housing 112.

A rear wall member embodied in this example by the rear cap 113 is axially movable from a lock position (see FIG. 6) where an inner wall of the rear cap 113 radially encloses an outer surface of each arm 130a, 130b to a release position (see FIG. 8) where the rear cap 113 is radially offset relative to the outer surface of each arm 130a, 130b.

In this embodiment, the actuator housing 112 comprises two radially flexible tabs 132a, 132b each having an axially directed surface 134a, 134b (provided by a small hook-like member best seen in FIG. 9) adjacent to a counter surface 135a, 135b of an engagement member—in the form of two indentations in the rear cap 113—to form an axial abutment preventing the rear cap 113 from moving into the release position.

Thus, as illustrated in FIG. 6, the axially directed surface 134a, 134b of the hook-like member of each radially flexible tab 132a, 132b moves into axial abutment contact with the counter surface 135a, 135b of the respective indentation in the rear cap 113, thereby holding the rear cap 113 in the lock position.

With reference in particular to FIG. 7, a release member embodied in this example by the needle cover 105 is configured to be axially moved into contact with radial protrusions 133a, 133b of the flexible tabs 132a, 132b to radially deflect the flexible tabs 132a, 132b, thereby radially offsetting the axially directed surfaces 134a, 134b of the hook-like members from the counter surfaces 135a, 135b of the indentations of the rear cap 113.

As a result, the radially flexible tabs 132a, 132b of the actuator housing 112 snaps out of locking engagement with the rear cap 113.

As illustrated in FIG. 8, upon the user further pushing the proximal end of the needle cover 105 against a site of the body for delivering the medicament, the rear cap 113 is axially moved in a distal direction from the lock position to the release position, in which the inner wall of the rear cap 113 no longer radially encloses the outer surface of each arm 130a, 130b but rather is radially offset relative to the outer surface of each arm 130a, 130b.

As a result, the arms 130a, 130b will no longer radially press against the spring-loaded plunger rod 110 in order to axially fix the spring-loaded plunger rod 110 relative to the arms 130a, 130b. The plunger rod 110 is thus released by the spring 111 expanding, and the medicament 109 is delivered via the syringe 106 to the user.

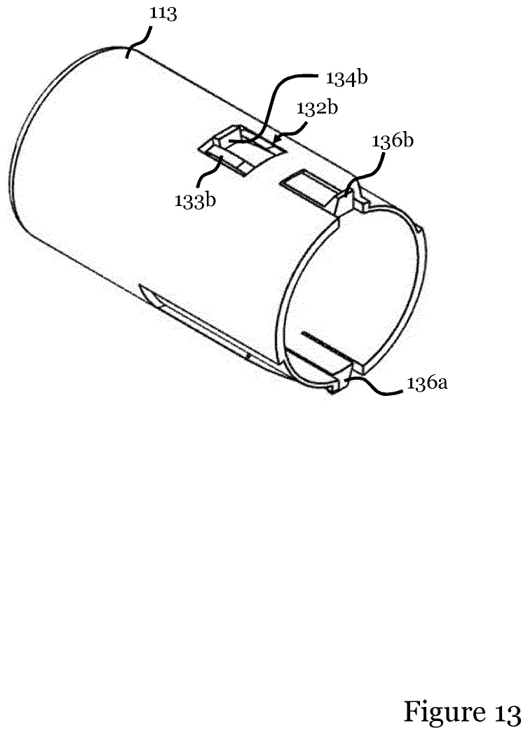

FIGS. 10-12 illustrate sectional views of the injector 100 of FIG. 3 taken along line A-A according to the second embodiment, while FIG. 13 shows the rear cap comprising the radially flexible tab in accordance with the second embodiment illustrated in FIG. 5. Only a few components of the exploded view of FIG. 5 will be discussed.

With reference to FIG. 10, in the sub-assembly of this embodiment, similar to the embodiments described with reference to FIGS. 6-9, the plunger rod 110 configured to be moveable inside the injector 100 is spring loaded by the spring 111 abutting the plunger rod 110 in one end and abutting an inner wall of the sub-assembly in another end, in this example a distal inner wall of the actuator housing 112, to compress the spring 111 upon the plunger rod 110 being moved towards a distal end of the injector 100.

Further, the sub-assembly comprises at least one arm (in this second exemplifying embodiment two arms 130a, 130b) extending axially and being immovable with respect to a housing. In an example where the sub-assembly is used with the medicament delivery device of FIG. 3, the housing is the back outer shell 101. The plunger rod 110 extends axially through an opening of the actuator housing 112 for contacting the arms 130a, 130b.

The arms 130a, 130b have a radially extending end section, an inner surface of which end section presses radially against the spring-loaded plunger rod 110 to axially fix the spring-loaded plunger rod 110 relative to the arms 130a, 130b.

A rear wall member embodied in this example by the rear cap 113 is axially movable from a lock position (see FIG. 10) where an inner wall of the rear cap 113 radially encloses an outer surface of each arm 130a, 130b to a release position (see FIG. 12) where the rear cap 113 is radially offset relative to the outer surface of each arm 130a, 130b.

In this embodiment, differing from that described with reference to FIGS. 6-9, the rear cap 113 (and not the actuator housing 112) comprises the two radially flexible tabs 132a, 132b each having an axially directed surface 134a, 134b (provided by a small hook-like member best seen in FIG. 13) adjacent to a counter surface 135a, 135b of an engagement member—in the form of two radially protruding stop members arranged at an interior of a side wall of the back outer shell 101—to form an axial abutment preventing the rear cap 113 from moving into the release position.

Thus, as illustrated in FIG. 10, the axially directed surface 134a, 134b of the hook-like member of each radially flexible tab 132a, 132b moves into axial abutment contact with the counter surface 135a, 135b of the respective stop member at the interior of the back outer shell 101, thereby holding the rear cap 113 in the lock position.

With reference in particular to FIG. 11, a release member embodied in this example by the needle cover 105 is configured to be axially moved into contact with the radial protrusions 133a, 133b of the flexible tabs 132a, 132b to radially deflect the flexible tabs 132a, 132b, thereby radially offsetting the axially directed surfaces 134a, 134b of the hook-like members from the counter surfaces 135a, 135b of the stop members of the back outer shell 101.

As a result, the radially flexible tabs 132a, 132b of the rear cap 113 snap out of locking engagement with the stop members of the back outer shell 101. The tab 132a will thus move under the stop member of the back outer shell 101 in a distal direction.

As illustrated in FIG. 12, upon the user further pushing the proximal end of the needle cover 105 against a site of the body for delivering the medicament, the rear cap 113 is axially moved in a distal direction from the lock position to the release position, in which the inner wall of the rear cap 113 no longer radially encloses the outer surface of each arm 130a, 130b but rather is radially offset relative to the outer surface of each arm 130a, 130b.

As a result, the arms 130a, 130b will no longer radially press against the spring-loaded plunger rod 110 in order to axially fix the spring-loaded plunger rod 110 relative to the arms 130a, 130b. The plunger rod 110 is thus released by the spring 111 expanding, and the medicament 109 is delivered via the syringe 106 to the user.

In the embodiment described with reference to FIGS. 10-14, since the radially flexible tabs 132, 132b are arranged on the rear cap 113 and not on the actuator housing 112, it may be envisaged that the actuator housing 112 is omitted. If so, the back outer shell 101 could be modified to comprise the arms 130a, 130b extending axially from an interior of an end wall of the back outer shell 101. Further, in such a scenario, a distal end of the spring 111 would abut the rear cap 113.

In a further embodiment, as illustrated throughout FIGS. 6-8 and 10-12, the plunger rod 110 may comprise an indentation received within the inner surface of the end section of the arms 130a, 130b.

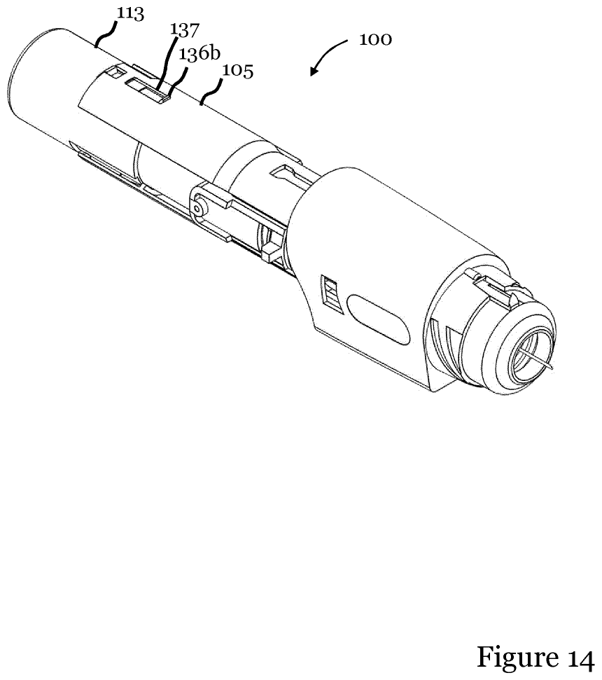

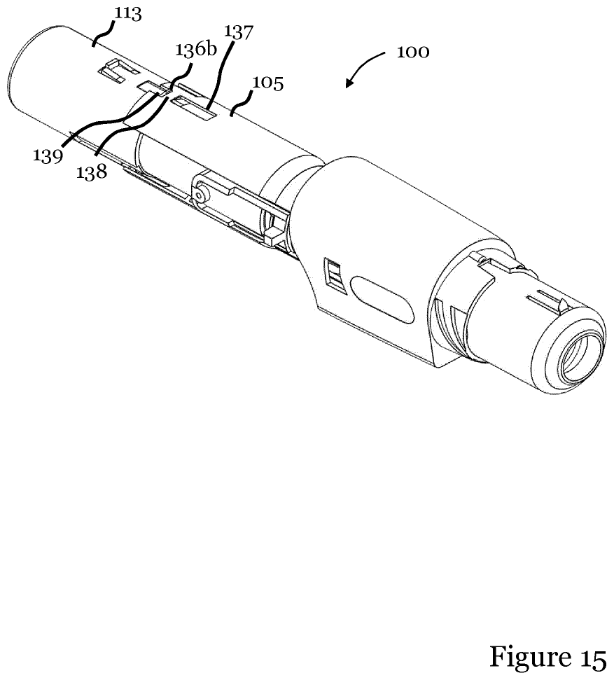

Further shown in FIG. 14 is that the rear cap 113 comprises radially protruding needle cover lock-out tabs 136a, 136b to be described in the following with further reference to FIGS. 14 and 15. With the lock-out tabs 136a, 136b, the lock-out collar 126 of FIG. 4 is not required.

FIG. 14 illustrates the injector 100 previously illustrated in FIG. 5 with the back outer shell removed. At this stage, the user has pressed the proximal end of the needle cover 105 against a site of the body for medicament delivery, wherein the rear cap 113 has been moved into the release position as described hereinabove.

As shown, the needle cover 105 comprises an axially extending guide slot 137 axially aligned with and configured to receive the needle cover lock-out tab 136b.

Initially, before the injector 100 is operated by the user, the lock-out tab 136b is positioned at a distal end of the guide slot 137. Upon the user pressing the proximal end of the needle cover 105 towards the body for medicament delivery to cause the needle cover 105 to move in a distal direction as shown in FIG. 14, a surface of a proximal end of the guide slot 137 is configured to move into axial abutment with an axially directed surface of a proximal side of the needle cover lock-out tab 137.

With reference to FIG. 15, the needle cover 105 comprises a wall section 138 distal of the guide slot 137, and a surface of a distal side of the lock-out tab 136b is configured to have an increasing inclination in a proximal direction.

Thus, upon the needle cover 105 reverting in a proximal direction to covering the needle of the injector 100 after completion of medicament delivery, the wall section 138 will slide over the inclining surface of the distal side of the lock-out tab 136b.

The axially directed surface of the proximal side of the lock-out tab 136b will move into axial abutment with a surface of a distal side of the wall section 138 if the needle cover 105 again is moved in a distal direction.

As shown, the needle cover 105 may comprise a lock-out tab slot 139 axially aligned with the guide slot 137 and located distal of the wall section 138 in the distal end of the needle cover 105.

The lock-out tab slot 139 is configured to accommodate the needle cover lock-out tab 136b upon the needle cover 105 moving in a proximal direction to shielding the needle.

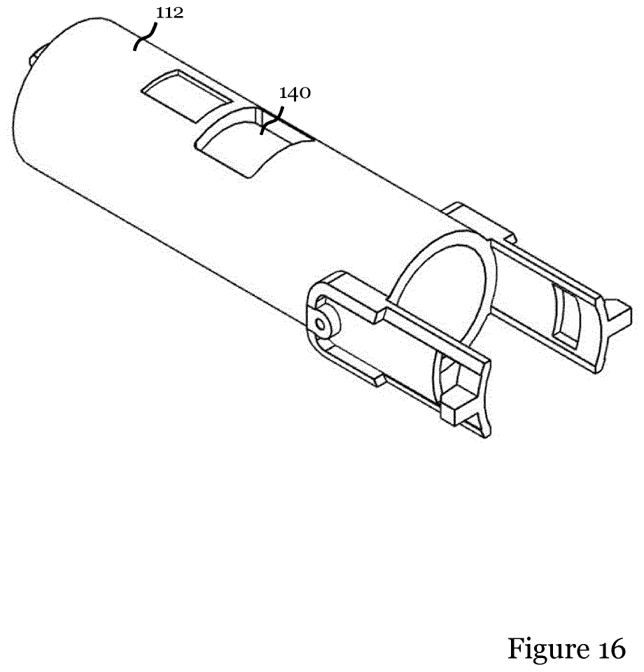

FIG. 16 illustrates that the actuator housing 112 comprises an outer wall opening 140 into which the lock-out tab 136b radially flexes when the wall section 138 of the needle cover 105 slides over the lock-out tab 136b.

Furthermore, the injector with the sub-assembly as disclosed in the second embodiment can be assembled with the following method.

First of all, the lock-out tab of the rear cap and the wall section of the needle cover are offset from each other. During assembly, guiding structures on both the rear cap and the needle cover interact to rotate the rear cap into an assembled position for the device to function properly. In one example, the guiding structures are a protrusion and a track. In a preferred example, the protrusion is arranged on an inner surface of the needle cover; and the track is a helical surface extending along the outer surface of the rear cap.

In a preferred example, the rear cap is installed on the actuator housing rotated approximately 30° about the axis A in a clockwise direction during assembly. During a step of the assembly, when the rear ca, the plunger rod, and the spring are inserted into the back outer shell, the lock out tab rests on the inside wall of the back outer shell.

During a final step of the assembly of the medicament delivery device, when the needle cover is assembled together with the rear outer shell, the guiding structure on the needle cover rotates the rear cap into a final assembled position. The guide slots of the needle cover align with the lock out tabs of the rear cap and the protrusion of the needle cover align with the helical surface of the rear cap. When the needle cover is pushed relative to the back outer shell during assembling, the protrusion of the needle cover travels along the helical surface of the rear cap, which causes the rear cap to rotate in the counter clockwise direction. As the rear cap rotates, the lock out tabs hit the wall of the guide slots of the needle cover, which forces them to flex inward and underneath the needle cover. When the protrusion of the needle cover reaches the end of the helical surface of the rear cap, the rear cap fully rotates into its final assembled position and the lock out tabs line up in the guide slot of the needle cover proximal to the wall section of the needle cover and flex back to their initial position.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The aspects of the present disclosure have mainly been described above with reference to a few embodiments and examples thereof. However, as is readily appreciated by a person skilled in the art, other embodiments than the ones disclosed above are equally possible within the scope of the invention, as defined by the appended patent claims.

Thus, while various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1-14. (canceled)

15. A sub-assembly of a medicament delivery device comprising:
a tubular housing, a plunger rod, a spring, a rear wall member, a radially flexible tab, an arm, and a release member,
wherein the plunger rod is configured to be moved by the spring inside the housing to expel a medicament of the medicament delivery device,
wherein the arm is axially immovable with respect to the housing, the arm having a radially extending end section, an inner surface of which end section pressing radially against the plunger rod to axially fix the plunger rod relative to the arm,
wherein the rear wall member is axially movable from a lock position where an inner wall of the rear wall member radially encloses an outer surface of the arm to a release position where the rear wall member is radially offset relative to the outer surface of the arm,
wherein the radially flexible tab comprises an axially directed surface adjacent to a counter surface of an engagement member to form an axial abutment preventing the rear wall member from moving into the release position,
wherein the release member is configured to be axially moved into contact with a radial protrusion of the flexible tab to radially deflect the flexible tab, thereby radially offsetting the axially directed surface from the counter surface of the engagement member.

16. The sub-assembly of claim 15, wherein
the release member is configured to be further axially moved to move the rear wall member to the release position after the axially directed surface has been radially offset from the counter surface of the engagement member.

17. The sub-assembly of claim 15, further comprising:
an actuator housing attached within the housing, wherein the actuator housing comprises an end wall with an interior abutting a distal end of the spring, and an opening via which the plunger rod extends for contacting the arm, the actuator housing further comprising the radially flexible tab and being immovable with respect to the plunger rod upon the rear wall member being in the lock position, and wherein the rear wall member comprises the engagement member

18. The subassembly of claim 17, wherein
the actuator housing comprises the arm extending axially from an exterior of the end wall of the actuator housing.

19. The subassembly of claim 15, wherein the housing comprises the arm extending axially from an interior of an end wall of the housing.

20. The sub-assembly of claim 15, wherein the plunger rod comprises an indentation; and wherein the indentation is received within the inner surface of the end section of the arm.

21. The sub-assembly of claim 15, wherein the rear wall member comprises the radially flexible tab, and wherein the housing comprises the engagement member arranged at an interior of a side wall of the housing.

22. The sub-assembly of claim 15, wherein the release member comprises a needle cover.

23. The sub-assembly of claim 22, wherein the needle cover is configured to axially move into contact with the radial protrusion of the flexible tab to radially deflect the flexible tab upon a user pressing a proximal end of the needle cover towards a site of the body for medicament delivery.

24. The sub-assembly of claim 22, wherein the needle cover is configured to axially move into contact with the rear wall member to move the rear wall member axially relative to the arm upon a user pressing a proximal end of the needle cover towards a site of the body for medicament delivery.

25. A medicament delivery device comprising the sub-assembly of claim 15.

26. The medicament delivery device of claim 25, wherein the medicament delivery device is an auto-injector.

27. The medicament delivery device of claim 25, wherein the auto-injector is a needle-based injector or a needleless injector.

28. The medicament delivery device of claim 25, wherein the medicament delivery device comprises a medicament container that is immovable relative to the tubular housing.
